(19) United States
(12) Patent Application Publication
(10) Pub. No.: US 2024/0074691 A1
(43) Pub. Date: Mar. 7, 2024

(54) DEVICES, SYSTEM, AND METHODS FOR PERFORMING ELECTRORETINOGRAPHY

(71) Applicants: Machelle T. Pardue, Decatur, GA (US); CARA MOTZ, Decatur, GA (US); KYLE CHESLER, Decatur, GA (US)

(72) Inventors: Machelle T. Pardue, Decatur, GA (US); CARA MOTZ, Decatur, GA (US); KYLE CHESLER, Decatur, GA (US)

(21) Appl. No.: 17/766,947

(22) PCT Filed: Oct. 8, 2020
(86) PCT No.: PCT/US20/54781

§ 371 Date: Apr. 6, 2022

Related U.S. Application Data
(60) Provisional application No. 62/912,920, filed on Oct. 9, 2019.

(51) Int. Cl. A61B 5/398; A61B 5/00; A61B 5/297

(52) U.S. Cl. CPC A61B 5/398; A61B 5/297; A61B 5/6814; A61B 5/6831; A61B 5/7271; A61B 2560/02; A61B 2560/0406; A61B 2560/0468; A61B 2562/164

(57) ABSTRACT

A wearable device for administering an electroretinography examination to a wearer can have a housing that defines a first and a second compartment. Each of the first and second compartments can comprise: a stimulation light source, a focal light source, an active electrode that is configured to engage skin of the wearer, and a reference electrode that is spaced from the active electrode and configured to engage skin of the wearer. A processor can be communicatively coupled to the stimulation light source, the active electrode, and the reference electrode of each of the first and second compartments of the housing. A memory can be in communication with the processor. The device can perform a method comprising: causing the stimulation light source of the first compartment to flash; and storing a signal from the active electrode of the first compartment. The housing can further comprise a ground electrode.

### CROSS-REFERENCE TO RELATED APPLICATION

This application claims priority to and the benefit of U.S. Provisional Patent Application No. 62/912,920, filed Oct. 9, 2019, the entirety of which is hereby incorporated by reference herein.

### FIELD

This disclosure relates generally to systems, devices, and methods for testing for retinal disease.

### BACKGROUND

Diabetic retinopathy (DR) is a leading cause of blindness in working-age adults. Fundus photography is the conventional method for diagnosing DR. However, by the time DR is evident on the fundus photo, irreversible damage and vision loss have already occurred. Electroretinography (ERG) is a powerful tool for recording retinal function that is used in opththalmology clinics to diagnose or monitor retinal disease. By probing for defects in rod-driven pathways, the ERG can be used to screen for DR in its earlier stages before vision threatening damage is present. The utility of ERGs as a screening device to diagnose retinal diseases is hindered due to: (a) the need for a prolonged period of dark adaptation of the patient's eyes in a darkened room, which is required to probe different retinal cell types; (b) the size/footprint of an ERG system; and (c) the expertise needed to interpret the results. These characteristics have limited the use of the ERG in primary care settings.

ERGs are traditionally performed using a desktop system with a Ganzfeld dome, in which the patient places their face to receive a uniform flash stimulus to both eyes. A typical clinical ERG will follow the standards published by the International Society for Clinical Electrophysiology in Vision (ISCEV), which recommend pupil dilation followed by 20 minutes of dark adaptation prior to presenting test flashes that efficiently probe retinal response dominated by rod photoreceptors and 10 minutes of light adaptation to isolate cone photoreceptor responses. Responses to ERG flash stimuli can be recorded using contact lens or fiber electrodes that touch the cornea or skin electrodes placed below the eye. The recorded responses require expert analysis thereof to produce diagnostically relevant information. Recent advances in ERG include hand-held ERG systems that are more portable and can potentially be used outside the clinic. One contemporary testing device is the RETeval developed by LKC Technology, Inc. The RETeval is a hand held, portable ERG system used to evaluate retinal function. It contains a pupil tracker that eliminates the need for dilating drops to enlarge the pupil diameter. The RETeval is being used in clinical and preclinical ophthalmology research. For instance, the RETeval has been shown to accurately screen for diabetic retinopathy using noninvasive skin electrodes and a portable, handheld ERG system with similar or better sensitivity than current DR methods using fundus exams and to detect drug toxicity with similar reliability to desktop ERG models.

However, the RETeval device does have limitations. The RETeval is limited to evaluating one eye at a time—doubling the valuable clinic time necessary to assess retinal function in both eyes. When using standard ERG devices or the RETeval, the dark adaptation step must be carried out in a dark room in order to dark-adapt the patient prior to testing. Since very few clinics have a designated room with a revolving dark room door for the dark adaptation period, the clinician or technician performing the exam must also work in the dark doing high-dexterity tasks, like placing electrodes in the eye.

The ERGs taken by the RETeval require expert analysis and interpretation of the results to draw meaningful, diagnostic information from the recordings. This requires a highly trained professional to take precious time in a busy clinic for the analyses.

### SUMMARY

Described herein is a wearable device for administering an electroretinography examination to a wearer of the device. The wearable device can comprise a housing having a first side and a second side spaced apart relative to a transverse axis. The housing can define first and second compartments positioned along the transverse axis, each of the first and second compartments being configured for positioning over a respective eye of the wearer. Each of the first and second compartments can comprise a stimulation light source, a focal light source, wherein the focal light source is positioned at a location where the respective eye of the wearer is focused during administration of the electroretinography examination, an active electrode that is configured to engage skin of the wearer, and a reference electrode that is spaced from the active electrode and configured to engage skin of the wearer. At least one processor can be communicatively coupled to the stimulation light source, the active electrode, and the reference electrode of each of the first and second compartments of the housing. A memory can be in communication with the processor, wherein the memory comprises instructions that, when executed by the processor, perform a method comprising: causing the stimulation light source of the first compartment to flash; and storing a signal from the active electrode of the first compartment. The housing can further comprise a ground electrode.

The memory can comprise instructions that, when executed by the processor, perform a step of detecting at least one feature of the signal.

The memory can comprise instructions that, when executed by the processor, perform a step of determining a time delay between the flash of the stimulation light source of a time of the at least one feature of the signal.

The stimulation light sources of the first and second compartments can be configured to uniformly illuminate an entire field of view of each eye of a wearer.

The stimulation light sources of the first and second compartments can be configured to provide a dim flash having a single flash intensity.

The stimulation light sources of the first and second compartments can be configured to provide a plurality of flashes of varying intensity.

The wearable device can further comprise a head strap having a first end that is attached to the first side of the housing and a second end that is attached to the second side of the housing.

The housing can comprise a flexible rim that is configured to conform to a face of the wearer.

The housing can be configured to block out substantially all ambient light to eyes of the wearer.

The active electrodes and the reference electrodes of the first and second compartments and the ground electrode can be embedded within the rim.

The active electrode of the first compartment can be positioned to engage skin of the wearer below the respective eye of the wearer.

The ground electrode can be positioned to engage at least one of a forehead skin or a brow skin of the wearer.

The reference electrode of the first compartment can be spaced further from a plane that is perpendicular to the transverse axis and bisects the housing between the first side and the second side than the active electrode of the first compartment.

The wearable device can further comprise an output device, wherein the output device is one of a cable, wireless transmitter, and an I/O port.

The housing can define a slot between the first and second compartments that is configured to conform to the shape of a nose of the wearer.

A spacing between the focal light sources of the first and second compartments can be fixed.

A spacing between the focal light sources of the first and second compartments can be selectively adjustable.

Each of the first and second compartments can comprise: a peripheral interior wall that extends circumferentially around a respective eye of the wearer and a distal wall that extends between distal surfaces of the peripheral interior wall to enclose a space that is visible by the eye of the wearer, wherein the stimulation light source and focal light source are secured to the distal wall.

A method of using the wearable device can comprise positioning the wearable device over the eyes of a wearer, executing instructions in the memory that cause the wearable device to perform a electroretinography test, and receiving an output from the wearable device.

Executing instructions in the memory that cause the wearable device to perform an electroretinography test can comprise simultaneously performing an electroretinography test on each eye of the wearer.

The method can further comprise analyzing the output from the wearable device to determine whether the patient has diabetic retinopathy.

The instructions can be executed after an acclimation period of at least five minutes.

### BRIEF DESCRIPTION OF THE DRAWINGS

These and other aspects of the invention will become more apparent in the detailed description in which reference is made to the appended drawings wherein:

FIG. 1 is a front perspective view of a wearable device in accordance with embodiments disclosed herein.

FIG. 2 is a rear view of the wearable device of FIG. 1.

FIG. 3 is a schematic of the wearable device of FIG. 1.

FIG. 4 is a computing device for receiving and/or processing data from the wearable device.

FIG. 5 is a graph showing raw data collected with the wearable device of FIG. 1.

FIG. 6 is a graph showing the data of FIG. 5 after it has been filtered.

FIG. 7 is the graph of FIG. 6 with waveform features indicated.

FIG. 8 is a plot showing data of a healthy retina and an unhealthy retina.

FIG. 9 is a rear view of a wearable device in accordance with embodiments disclosed herein, with the locations of a wearer's eyes shown schematically.

### DETAILED DESCRIPTION

The present invention can be understood more readily by reference to the following detailed description, examples, drawings, and claims, and their previous and following description. However, before the present devices, systems, and/or methods are disclosed and described, it is to be understood that this invention is not limited to the specific devices, systems, and/or methods disclosed unless otherwise specified, and, as such, can, of course, vary. It is also to be understood that the terminology used herein is for the purpose of describing particular aspects only and is not intended to be limiting.

The following description of the invention is provided as an enabling teaching of the invention in its best, currently known embodiment. To this end, those skilled in the relevant art will recognize and appreciate that many changes can be made to the various aspects of the invention described herein, while still obtaining the beneficial results of the present invention. It will also be apparent that some of the desired benefits of the present invention can be obtained by selecting some of the features of the present invention without utilizing other features. Accordingly, those who work in the art will recognize that many modifications and adaptations to the present invention are possible and can even be desirable in certain circumstances and are a part of the present invention. Thus, the following description is provided as illustrative of the principles of the present invention and not in limitation thereof.

As used throughout, the singular forms “a,” “an,” and “the” include plural referents unless the context clearly dictates otherwise. Thus, for example, reference to “an electrode” can include two or more such electrodes unless the context indicates otherwise.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. Optionally, in some aspects, when values are approximated by use of the antecedents “approximately,” “about,” “generally,” or “substantially,” it is contemplated that values within up to 15%, up to 10%, up to 5%, or up to 1% (above or below) of the particularly stated value or characteristic can be included within the scope of those aspects.

As used herein, the terms “optional” or “optionally” mean that the subsequently described event or circumstance may or may not occur, and that the description includes instances where said event or circumstance occurs and instances where it does not.

As used herein, the term “communicatively coupled” refers to a condition in which two components are capable of communicating with each other using any conventional wired or wireless communication protocol, including, without limitation, direct/cable connection, Wi-Fi® connection, Bluetooth® connection, a radiofrequency (RF) communication protocol, and the like.

The word “or” as used herein means any one member of a particular list and also includes any combination of members of that list unless otherwise clear from the context.

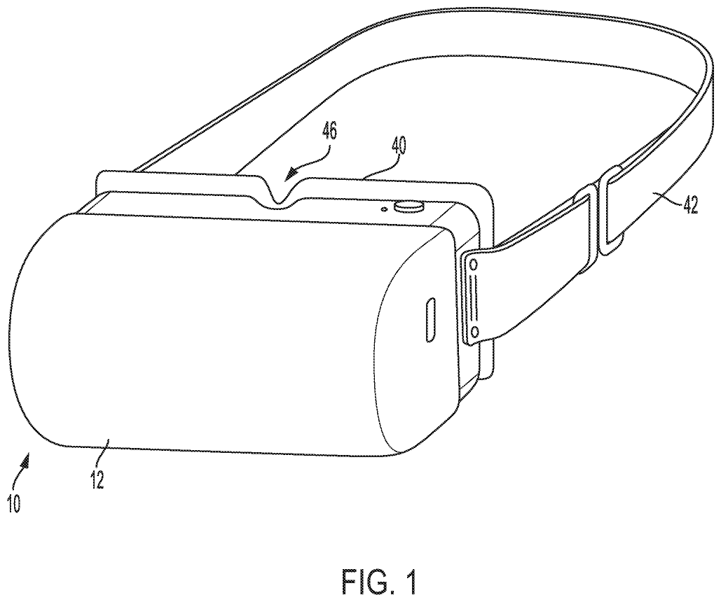

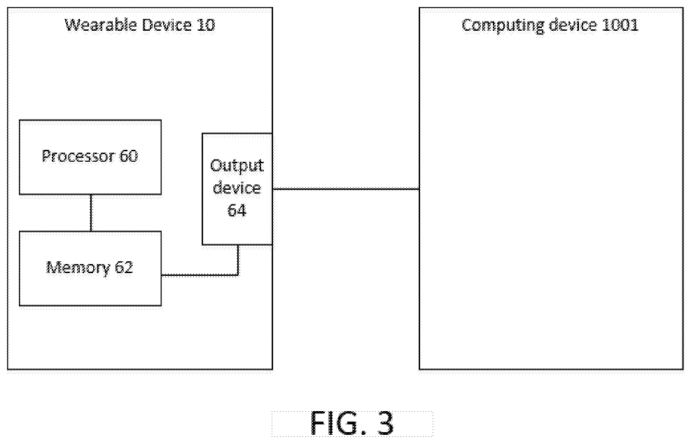

Described herein, with reference to FIGS. 1-4 and 9, are systems and methods for diagnosing retinal disease, such as diabetic retinopathy (DR). According to one aspect, a wearable, non-invasive diagnostic system can use ERG to detect DR. The system can include a compact device that is wearable as a pair of goggles (or other suitable eye coverings). The device can include at least one electrode (optionally, a plurality of electrodes). The electrodes can be disposed in the rim of the googles and be are configured to receive and record electrical signals indicative of electrical function of the eye via contact with skin around the eye.

The system can fully cover both eyes so that dark adaptation can occur in a controlled manner under a variety of settings (e.g., in a lighted or partially lighted room), allowing the technician to work and conduct testing while the room remains illuminated. Thus, in contrast to conventional ERG systems, it is contemplated that the systems, devices, and methods disclosed herein can be used without the need for a dark room. According to some aspects, the system can screen for DR using a prescribed dim flash (e.g., a single period or interval of flashing) to probe for rod-driven pathways that are affected in DR, negating the need for long, multistep flash protocols employed by conventional ERG systems. In these aspects, it is contemplated that the flash transmission of the disclosed system can consist of a single interval or period of stimulation. In further aspects, the system can automatically process and analyze recordings to provide diagnostic information. The disclosed system can remedy the current shortcomings of clinical ERG that hinder the use of ERG as a standard, routine screening device for DR in the primary care or ophthalmology clinic.

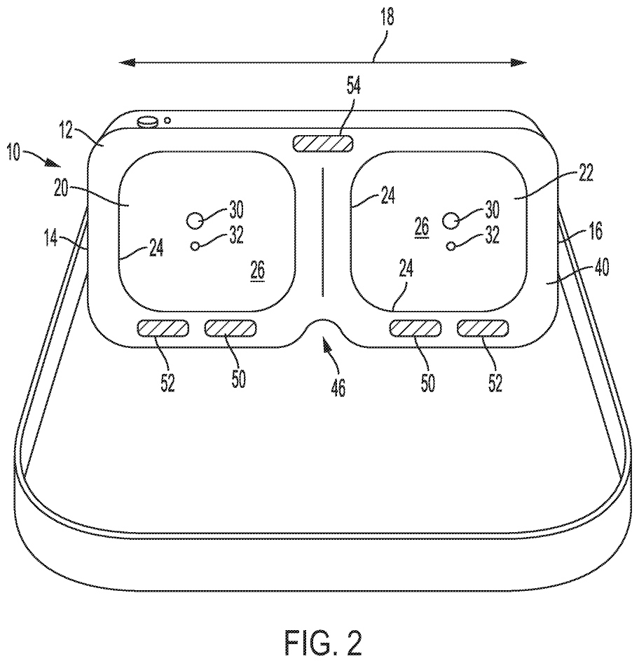

An electroretinography system in accordance with embodiments disclosed herein can comprise a wearable device 10 comprising a housing 12 having a first side 14 (shown in FIG. 2 as the left side of the device from the perspective of the wearer) and a second side 16 (shown in FIG. 2 as the right side of the device from the perspective of the wearer). The first side 14 and second side 16 can be spaced apart relative to a transverse axis 18. The housing 12 can define a first compartment 20 on the first side 14 and a second compartment 22 on the second side 16. The first and second compartments can be sized and spaced for positioning over respective eyes of a wearer. For example, according to some aspects, the distance between the centers of the first and second compartments can be approximately the average pupillary distance (PD) between an adult's eyes. In these aspects, it is contemplated that the distance between the centers of the first and second compartments can range from about 58 mm to about 68 mm. Each compartment can comprise a peripheral interior wall 24 that extends circumferentially around a respective eye of the wearer (when in an operative/use position) and projects outwardly (away from the eye) in a distal direction. A distal wall 26 can extend between distal ends (e.g., distal edges or surfaces) of the peripheral interior wall 24 such that a respective eye of the wearer faces the distal wall 26 when the device is in the operative/use position. The interior peripheral wall 24 and distal wall 26 can cooperate to enclose a space that is visible by the eye of the wearer. Although described above as forming distinct sections of the compartment, it is contemplated that the peripheral interior wall 24 and the distal wall 26 can cooperate to define a rounded compartment profile that does not include a defined separation between the two walls.

Each compartment can comprise a stimulation light source 30 and a focal light source 32. In some optional aspects, the focal light source 32 can emit red light. In further optional aspects, the stimulation light source 30 can emit white light (for example, a light transmission consisting of white light). Optionally, the stimulation light sources 30 and focal light sources 32 can be secured to the distal walls 26 of the respective compartments 20, 22. Optionally, the housing can define a respective slot in each compartment into which the respective stimulation light sources 30 and/or focal light sources 32 can be embedded. The housing 12 can cooperate with each stimulation light source 30 to produce a Ganzfeld dome over each eye of the wearer. That is, the stimulation light source 30 can be configured to uniformly, or substantially uniformly, illuminate an entire field of view of each eye of the wearer. This can be accomplished with a housing having a sufficient depth and compartments that comprise or are covered with Ganzfeld paint (e.g., frosted paint) that reflects the light within the respective compartment or a plastic that diffuses light evenly. Optionally, the stimulation light source 30 can comprise a filter that is configured to create a diffuse light source as disclosed herein. In exemplary aspects, the color of the stimulation light source 30 will not be controlled. The focal light sources 32 can optionally be centered in their respective compartments so that as the stimulation light source 30 produces a flash, the flash can bounce off the respective compartment's walls and distribute evenly to the retina of the respective eye of the wearer. In some embodiments, the spacing between the focal light sources 32 of the first and second compartments 20, 22 can be fixed. In other embodiments, the spacing between the focal light sources 32 of the first and second compartments 20, 22 can be adjustable. For example, it is contemplated that each compartment can comprise respective tracks or a series of mounting locations that permit adjustment of the positions of each focal light source 32. Optionally, the spacing between the first and second compartments themselves can be adjustable. For example, the device can comprise a nose bridge (as is known in other types of goggles) that permits selective adjustment of the spacing between the two compartments. In this example, it is contemplated that the compartments can be formed as separate components that are coupled together by the nose bridge.

The housing can comprise a rim 40 that can extend around the circumference of each compartment. Optionally, the rim 40 can comprise a flexible polymer (e.g., silicone or rubber) that can be configured to resiliently conform to the wearer's face so that the housing blocks out most, substantially all, or all ambient light from entering the wearer's field of view. In this way, the device can be used in a lit or partially lit room, thereby allowing a medical professional to see during setup and examination while allowing the wearer's eyes to dark-adapt. The rim 40 can comprise a material that can be sanitized with an alcohol prep pad. The rim 40 can be removable for sanitation or replacement. A band 42 (optionally, an elastic band) can attach at a first end to the first side of the housing and at a second end to the second side of the housing. The band 42 can extend around the wearer's head to secure the device 10 to the wearer. The band 42 can optionally be adjustable via known means (e.g., buckles, slip locks, and other adjuster elements) to provide a comfortable fit, yet applying the housing 12 to the wearer's face at a pressure that causes the rim 40 to conform to the wearer's face to sufficiently block out ambient light. The band 42 can attach to the housing via a snap closure to allow the band to be removed for cleaning or replacement. The housing can define a slot 46 between the first and second compartments 20, 22 that is configured to conform or substantially conform to, or be complementary to, the shape of the wearer's nose.

An active electrode 50 and a reference electrode 52 can be disposed in the rim at each of the first compartment 20 and the second compartment 22. Optionally, the active electrodes and reference electrodes 52 can be disposed in the rim to engage skin just below the wearer's eye. The active electrodes 50 can be spaced from the respective reference electrodes 52. The reference electrodes 52 can be spaced farther from the eye than the respective active electrodes 50. Optionally, the active electrode 50 can be spaced inwardly of the respective reference electrode 52 (i.e., closer to the compartment for the contralateral eye) relative to the transverse axis 18. That is, the reference electrode 52 of each compartment can be spaced further from a plane 36 that is perpendicular to the transverse axis 18 and bisects the housing between the first side and the second side than the active electrode 50 of the respective compartment. A ground electrode 54 can be disposed in the rim 40 of the housing 12. Optionally, the ground electrode 54 can be positioned in the rim 40 to engage the wearer's brow skin or forehead skin. Optionally, it is contemplated that the ground electrode 54 can be approximately centrally positioned along the transverse axis 18 such that the ground electrode 54 is intersected by the reference plane 36. Thus, as shown in FIG. 2, it is contemplated that the ground electrode 54 can be disposed in an upper portion of the rim 40 (above the eyes of the wearer), while the active and reference electrodes 50, 52 of each compartment are disposed in a lower portion of the rim (below the eyes of the wearer).

In exemplary aspects, the rim can define respective receptacles that receive at least a portion of a corresponding active, reference, or ground electrode, with each receptacle defining an opening that permits direct contact between the electrode and the skin of the wearer. Additionally, or alternatively, the rim can define at least one receptacle that receives a plurality of active, reference, or ground electrodes. Optionally, the electrodes can be adhesively secured within each receptacle. Alternatively, the electrodes can be mechanically retained within each receptacle (e.g., at least in part by portions of the rim that extend over the electrode). In still further aspects, it is contemplated that the In further aspects, it is contemplated that the rim and the housing of the wearable device 10 can cooperate to define structure that accommodates any circuitry components that are electrically connected to the various electrodes provided within the rim.

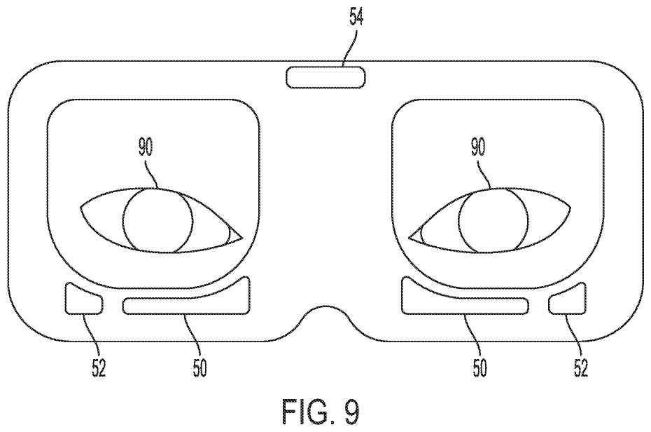

Referring also to FIG. 9 and as shown with reference to eye locations 90, the active electrodes 50 can be placed in the rim to minimize distance from the respective eyes of the wearer. The active electrodes 50 can have a horizontal elongate profile to help to maximize the skin-electrode contact surface area. In exemplary aspects, the active electrodes 50 can be longer than the corresponding reference electrodes (measured relative to the transverse axis 18). The reference electrodes 52 can be under the eyes and spaced closer to a respective side of the goggles than the respective active electrode. The ground electrode 54 can be at the top of the goggle-skin interface and configured to contact the center of the lower forehead. The electrodes can be flat, thin sheets of metal, similar to foil. The electrodes can optionally be flexible. The edges of the electrodes can be covered by the rim so that no sharp edges are exposed to the wearer. The electrodes can protrude from the rim toward the wearer by about 0.1 mm to about 1 mm (optionally, about 0.5 mm) to facilitate contact with the skin of the wearer. Optionally, in exemplary aspects, it is contemplated that the overall configuration and arrangement of the active and reference electrodes 50, 52 can be symmetrical or generally symmetrical relative to a plane that bisects the goggles in between the first and second compartments.

The device 10 can comprise a processor 60 (or a plurality of processors) that is communicatively coupled to the stimulation light source 30, the active electrode 50, and the reference electrode 52 of each of the first and second compartment as well as the ground electrode 54 and, optionally, the focal light source 32 of each compartment. A memory 62 can be in communication with the processor 60. The memory 62 can comprise instructions for performing an ERG test on at least one eye. For example, the instructions can cause the stimulation light of the first compartment to flash and then cause the device 10 to store one or more signals received from the active electrode from the first compartment. The memory 62 can optionally provide instructions for simultaneously performing an ERG test on the contralateral eye. For example, the instructions can cause the stimulation light of the second compartment to flash and then cause the device to store one or more signals received from the active electrode from the second compartment. As should be understood, simultaneously performing a test (on both eyes) should not be limited to simultaneous flashing of the stimulation lights, although, in some embodiments, simultaneous flashing can be used. However, in further embodiments, simultaneous testing can merely refer to the device 10 performing a series of ERG tests on the wearer's first eye with the first compartment while, during the same duration, performing a series of ERG tests on the wearer's second eye with the second compartment.

The device 10 can be configured to perform an ERG test comprising a dim flash test. In some embodiments, the flash duration can be less than 0.5 milliseconds. In some embodiments, the ERG test can comprise flashes of only one single intensity. In this way, the device 10 can be configured for one type of screening. In further embodiments, the test can comprise a plurality of flash intensities. In these embodiments, it is contemplated that the plurality of flash intensities can be delivered in a desired sequence or pattern. In exemplary aspects, the flash test can consist of a single sequence or pattern of flashes.

The device 10 can be in communication with a remote computing device 1001, such as, for example, a desktop computer, tablet, or smartphone. For example, the device 10 can communicate via an output device 64 such as a cable, an I/O port, or a wireless transmitter. The device 10 and remote computing device 1001 can communicate via any protocol, including, but not limited to, RS-232, Wi-Fi, RF, and Bluetooth.

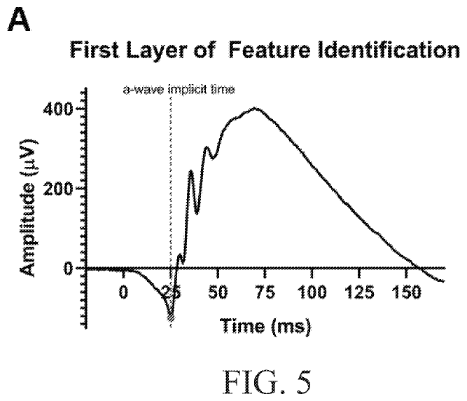

The data from the ERG tests can further be processed, for example, to determine whether or not the wearer has diabetic retinopathy. In some embodiments, the remote computing device 1001 can perform the data processing. Optionally, in further embodiments, the processor 60 can be configured to perform at least some (or, optionally, all) of the data processing. For example, the processor can extract abstract waveform features that can be used to diagnose early retinal dysfunction associated with diabetic retinopathy. Referring to FIGS. 5-8, an algorithm can extract an implicit time of an a-wave from the raw data. The raw data can comprise electrical potential (in microvolts) measured by the electrodes as a function of time. The implicit time can indicate the duration of time between the beginning of the flash stimulus and a waveform feature (such as an a-wave, b-wave, or oscillatory potential). Such waveform features can optionally be local maxima and minima of the raw data (or raw data filtered by a low pass filter) and can be extracted via software that finds said maxima and minima. For example, the software can find the first derivative of the raw data (or filtered raw data), or a curve through the raw data (or filtered raw data), and can determine potential waveform features where the first derivative changes from a positive to a negative. Peaks of the potential waveform features can be compared to a threshold to determine, based on their respective amplitudes and widths, whether the potential waveform feature is a waveform feature or noise. In further aspects, a search algorithm can search for maximum or minimum data values to find said local maxima and minima.

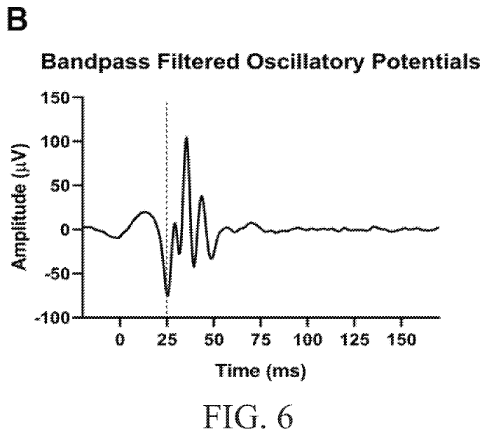

Optionally, signal smoothing and/or band pass filters can be used to reduce noise of, or enhance features from, the raw data for data processing. For example, as shown in FIG. 6, the data can be passed through one or more digital bandpass filters to isolate and output an oscillatory potentials (OPs) waveform.

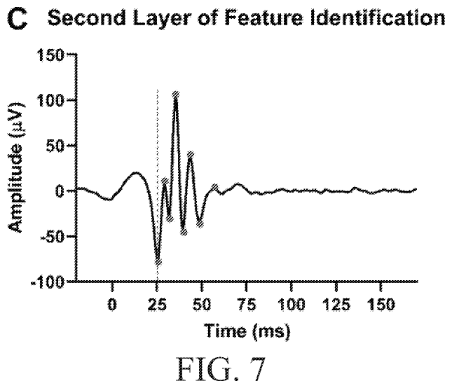

Referring to FIG. 7, the implicit time and OPs waveform can be input into a second layer of the algorithm. Based on a function of the a-wave implicit time and one or more waveform characteristics (e.g., height, prominence, and width of the waveform features), additional waveform features such as, for example, OPs, can be identified. The a-wave implicit time can be used as a basis to begin searching for OPs. For example, a search algorithm can begin searching for OPs after the a-wave implicit time, since the waveform features cannot be before the a-wave implicit time. As disclosed above, the first derivative of the (raw or filtered) data can be searched to find local minima and maxima of the data. The maxima and minima can be compared to absolute and relative amplitude and width thresholds to extract waveform features (e.g., OPs) from local maxima and minima caused by noise.

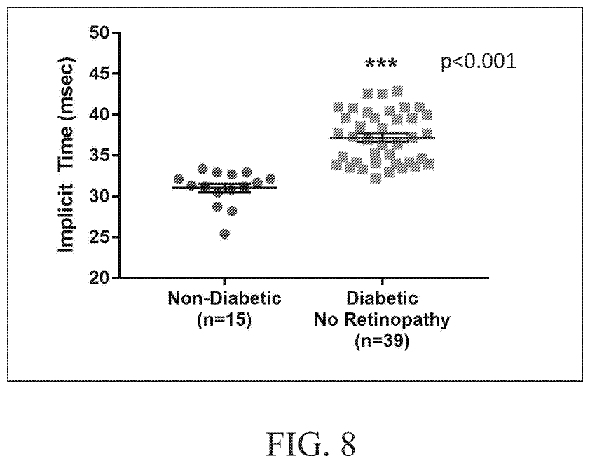

The time delays from the stimulation flash to the waveform features can indicate whether the retina is healthy or unhealthy. For example, FIG. 8 illustrates delays in ERG oscillatory potential waveforms in patients without and patients with diabetes but with no signs of retinopathy in their fundus. The patients of the data shown in FIG. 8 were age-matched and had no signs of ocular disease. Optionally, the time delays associated with particular waveform features can automatically be compared to reference values (corresponding to recorded time delays associated with both healthy and unhealthy patients) to determine whether a patient's retina is healthy or suffers from an ailment (e.g., DR), and the device can output a binary (e.g., positive/negative) diagnosis. In these optional aspects, it is contemplated that the reference values can be stored in the memory (optionally, as a database) and retrieved by the processor to perform the comparison. In further embodiments, the data can be output to a monitor (e.g., of a remote computing device) for a medical professional to review. For example, the waveform features' time delays can be provided to the professional, and the professional can give a diagnosis. In some embodiments, it is contemplated that the device can provide an inconclusive diagnosis when the comparison to historical data does not produce a clear indication of the health of the patient's retina. According to further aspects, data collected by the device 10 can be used to test for drug toxicity, optionally using data capture and analysis methods similar to those described herein.

To use the device 10, a medical professional or the wearer can position the device over the wearer's eyes. The band 42 can be adjusted until the device is comfortable while blocking substantially all ambient light from reaching the wearer's eyes. The wearer can wait for an acclimation period until the wearer's eyes have adjusted to the darkness. The acclimation period can be at least five minutes, at least ten minutes, at least twenty minutes, or more. The professional can then cause the device 10 to execute the instructions in the memory 62 to begin the test. In further embodiments, the acclimation period can be part of the instructions, wherein the instructions include a delay period to allow for acclimation, and the device does not begin testing until the acclimation period has passed. Optionally, the device can perform an electroretinography test on both eyes simultaneously. Alternatively, the device can sequentially perform an electroretinography test on each eye without removing the device from the eyes of the wearer. The device 10, the remote computing device 1001, or a medical professional can then analyze the data collected by the test. For example, the device can output a positive or negative diagnosis on a display (e.g., an embedded display on the device or a remote display such as one in communication with the remote computing device 1001). In further embodiments, the computing device 1001 can receive data from the device 10 and process the data as disclosed herein. In still further embodiments, the professional can analyze the data. The device can further output an error signal if the recording could not be made, or if the data is unusable, e.g., due to excessive noise often caused by electrodes not making correct contact with the wearer.

The disclosed systems and methods provide various advantages over conventional electroretinography methods. The device 10 can assess both eyes simultaneously, negating the need for repeat testing in the contralateral eye and reducing testing time. Further, the patient can dark adapt simply by wearing the device, negating the need for a dark room in the clinic such that the clinician or technician does not have to work in the dark. This can allow testing to be done outside the eye clinic in a variety of settings, whether in primary care offices or hospitals that are not equipped with a traditional ERG system, or remote, underserved areas that lack specialty clinics and physicians, or transportation. The device 10 can automatically process and analyze ERG recordings within the device, reporting diagnostically relevant information without the need for expert analyses. Unlike existing devices, the disclosed device 10 can provide stimulation light flashes that do not substantially alter pupil size, thereby avoiding the need for providing a camera in the device or otherwise monitoring pupil size.

### Computing Device

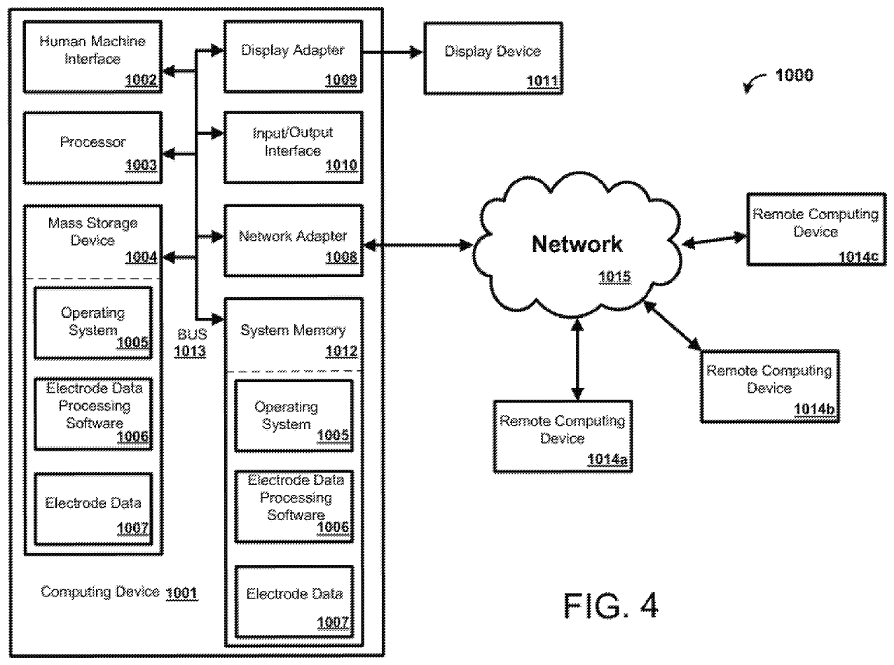

FIG. 4 shows a system 1000 including an exemplary configuration of a computing device 1001 for use with the device 10. The processor 60 and memory 62 can have a structure that is consistent with the structure of the computing device 1001. Moreover, in some embodiments, all of the aspects disclosed herein with respect to a separate computing device 1001 can be integrated within the device 10 so that the device 10 can perform all of the functions from setup of initial test parameters, starting the test, control of the test, processing of data, and diagnosis output. In further embodiments, a separate computing device 1001 can interface with the device 10 to control some or all of the ERG testing and analysis disclosed herein. In still further aspects, it is contemplated that the computing device 1001 can communicate with and cooperate with a remote computing device 1014 to control or perform one or more portions of the ERG analysis disclosed herein.

The computing device 1001 may comprise one or more processors 1003, a system memory 1012, and a bus 1013 that couples various components of the computing device 1001 including the one or more processors 1003 to the system memory 1012. In the case of multiple processors 1003, the computing device 1001 may utilize parallel computing.

The bus 1013 may comprise one or more of several possible types of bus structures, such as a memory bus, memory controller, a peripheral bus, an accelerated graphics port, and a processor or local bus using any of a variety of bus architectures.

The computing device 1001 may operate on and/or comprise a variety of computer readable media (e.g., non-transitory). Computer readable media may be any available media that is accessible by the computing device 1001 and comprises, non-transitory, volatile and/or non-volatile media, removable and non-removable media. The system memory 1012 has computer readable media in the form of volatile memory, such as random access memory (RAM), and/or non-volatile memory, such as read only memory (ROM). The system memory 1012 may store data such as electrode data 1007 (i.e., data from signals received by the electrodes) and/or program modules such as operating system 1005 and electrode data processing software 1006 that are accessible to and/or are operated on by the one or more processors 1003.

The computing device 1001 may also comprise other removable/non-removable, volatile/non-volatile computer storage media. The mass storage device 1004 may provide non-volatile storage of computer code, computer readable instructions, data structures, program modules, and other data for the computing device 1001. The mass storage device 1004 may be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like.

Any number of program modules may be stored on the mass storage device 1004. An operating system 1005 and electrode data processing software 1006 may be stored on the mass storage device 1004. One or more of the operating system 1005 and electrode data processing software 1006 (or some combination thereof) may comprise program modules and the electrode data processing software 1006. Electrode data 1007 may also be stored on the mass storage device 1004. Electrode data 1007 may be stored in any of one or more databases known in the art. The databases may be centralized or distributed across multiple locations within the network 1015.

A user (e.g., the medical professional) may enter commands and information into the computing device 1001 using an input device (not shown). Such input devices comprise, but are not limited to, a keyboard, pointing device (e.g., a computer mouse, remote control), a microphone, a joystick, a scanner, tactile input devices such as gloves, and other body coverings, motion sensor, and the like. These and other input devices may be connected to the one or more processors 1003 using a human machine interface 1002 that is coupled to the bus 1013, but may be connected by other interface and bus structures, such as a parallel port, game port, an IEEE 1394 Port (also known as a Firewire port), a serial port, network adapter 1008, and/or a universal serial bus (USB).

A display device 1011 may also be connected to the bus 1013 using an interface, such as a display adapter 1009. It is contemplated that the computing device 1001 may have more than one display adapter 1009 and the computing device 1001 may have more than one display device 1011. A display device 1011 may be a monitor, an LCD (Liquid Crystal Display), light emitting diode (LED) display, television, smart lens, smart glass, and/or a projector. In addition to the display device 1011, other output peripheral devices may comprise components such as speakers (not shown) and a printer (not shown) which may be connected to the computing device 1001 using Input/Output Interface 1010. Any step and/or result of the methods may be output (or caused to be output) in any form to an output device. Such output may be any form of visual representation, including, but not limited to, textual, graphical, animation, audio, tactile, and the like. The display 1011 and computing device 1001 may be part of one device, or separate devices.

The computing device 1001 may operate in a networked environment using logical connections to one or more remote computing devices 1014a,b,c. A remote computing device 1014a,b,c may be a personal computer, computing station (e.g., workstation), portable computer (e.g., laptop, mobile phone, tablet device), smart device (e.g., smartphone, smart watch, activity tracker, smart apparel, smart accessory), security and/or monitoring device, a server, a router, a network computer, a peer device, edge device or other common network node, and so on. Logical connections between the computing device 1001 and a remote computing device 1014a,b,c may be made using a network 1015, such as a local area network (LAN) and/or a general wide area network (WAN). Such network connections may be through a network adapter 1008. A network adapter 1008 may be implemented in both wired and wireless environments. Such networking environments are conventional and commonplace in dwellings, offices, enterprise-wide computer networks, intranets, and the Internet. It is contemplated that the remote computing devices 1014a,b,c can optionally have some or all of the components disclosed as being part of computing device 1001.

Application programs and other executable program components such as the operating system 1005 are shown herein as discrete blocks, although it is recognized that such programs and components may reside at various times in different storage components of the computing device 1001, and are executed by the one or more processors 1003 of the computing device 1001. An implementation of electrode data processing software 1006 may be stored on or sent across some form of computer readable media. Any of the disclosed methods may be performed by processor-executable instructions embodied on computer readable media.

Exemplary Aspects

In view of the described devices, systems, and methods and variations thereof, herein below are described certain more particularly described aspects of the invention. These particularly recited aspects should not however be interpreted to have any limiting effect on any different claims containing different or more general teachings described herein, or that the “particular” aspects are somehow limited in some way other than the inherent meanings of the language literally used therein.

Aspect 1: A wearable device for administering an electroretinography examination to a wearer of the device, the wearable device comprising: a housing having a first side and a second side spaced apart relative to a transverse axis, the housing defining first and second compartments positioned along the transverse axis, each of the first and second compartments being configured for positioning over a respective eye of the wearer and comprising: a stimulation light source, a focal light source, wherein the focal light source is positioned at a location where the respective eye of the wearer is focused during administration of the electroretinography examination, an active electrode that is configured to engage skin of the wearer, and a reference electrode that is spaced from the active electrode and configured to engage skin of the wearer; at least one processor communicatively coupled to the stimulation light source, the active electrode, and the reference electrode of each of the first and second compartments of the housing; and a memory in communication with the processor, wherein the memory comprises instructions that, when executed by the processor, perform a method comprising: causing the stimulation light source of the first compartment to flash; and storing a signal from the active electrode of the first compartment, wherein the housing further comprises a ground electrode.

Aspect 2: The wearable device of aspect 1, wherein the memory comprises instructions that, when executed by the processor, perform a step of detecting at least one feature of the signal.

Aspect 3: The wearable device of claim 2, wherein the memory comprises instructions that, when executed by the processor, perform a step of determining a time delay between the flash of the stimulation light source and at least one feature of the signal.

Aspect 4: The wearable device of any one of the preceding aspects, wherein the stimulation light sources of the first and second compartments are configured to uniformly illuminate an entire field of view of each eye of a wearer.

Aspect 5: The wearable device of any one of the preceding aspects, wherein the stimulation light sources of the first and second compartments are configured to provide a dim flash having a single flash intensity.

Aspect 6: The wearable device of any one of the preceding aspects, wherein the stimulation light sources of the first and second compartments are configured to provide a plurality of flashes of varying intensity.

Aspect 7: The wearable device of any one of the preceding aspects, wherein the wearable device further comprises a head strap having a first end that is attached to the first side of the housing and a second end that is attached to the second side of the housing.

Aspect 8: The wearable device of any one of the preceding aspects, wherein the housing comprises a flexible rim that is configured to conform to a face of the wearer.

Aspect 9: The wearable device of aspect 8, wherein the housing is configured to block out substantially all ambient light to eyes of the wearer.

Aspect 10: The wearable device of aspect 8 or aspect 9, wherein the active electrodes and the reference electrodes of the first and second compartments and the ground electrode are embedded within the rim.

Aspect 11: The wearable device of any one of the preceding aspects, wherein the active electrode of the first compartment is positioned to engage skin of the wearer below the respective eye of the wearer.

Aspect 12: The wearable device of any one of the preceding aspects, wherein the ground electrode is positioned to engage at least one of a forehead skin or a brow skin of the wearer.

Aspect 13: The wearable device of any one of the preceding aspects, wherein the reference electrode of the first compartment is spaced further from a plane that is perpendicular to the transverse axis and bisects the housing between the first side and the second side than the active electrode of the first compartment.

Aspect 14: The wearable device of any one of the preceding aspects, further comprising an output device, wherein the output device is one of a cable, wireless transmitter, and an I/O port.

Aspect 15: The wearable device of any one of the preceding aspects, wherein the housing defines a slot between the first and second compartments that is configured to conform to the shape of a nose of the wearer.

Aspect 16: The wearable device of any one of the preceding aspects, wherein a spacing between the focal light sources of the first and second compartments is fixed.

Aspect 17: The wearable device of any one of the preceding aspects, wherein a spacing between the focal light sources of the first and second compartments is selectively adjustable.

Aspect 18: The wearable device of any one of the preceding aspects, wherein each of the first and second compartments comprises: a peripheral interior wall that extends circumferentially around a respective eye of the wearer; and a distal wall that extends between distal surfaces of the peripheral interior wall to enclose a space that is visible by the eye of the wearer, wherein the stimulation light source and focal light source are secured to the distal wall.

Aspect 19: A method of using the wearable device of any of aspects 1-18, the method comprising: positioning the wearable device over the eyes of a wearer; executing instructions in the memory that cause the wearable device to perform a electroretinography test; and receiving an output from the wearable device.

Aspect 20: The method of aspect 19, wherein executing instructions in the memory that cause the wearable device to perform an electroretinography test comprises simultaneously performing an electroretinography test on each eye of the wearer.

Aspect 21: The method of aspect 19, further comprising analyzing the output from the wearable device to determine whether the patient has diabetic retinopathy.

Aspect 22: The method of any of aspects 19-21, wherein the instructions are executed after an acclimation period of at least five minutes.

Aspect 23: A wearable device for administering an electroretinography examination to a wearer of the device, the wearable device comprising: a housing having a first side and a second side spaced apart relative to a transverse axis, the housing defining first and second compartments positioned along the transverse axis, each of the first and second compartments being configured for positioning over a respective eye of the wearer and comprising: a stimulation light source, a focal light source, wherein the focal light source is positioned at a location where the respective eye of the wearer is focused during administration of the electroretinography examination, an active electrode that is configured to engage skin of the wearer, and a reference electrode that is spaced from the active electrode and configured to engage skin of the wearer, wherein the housing is configured to block substantially all ambient light from the eyes of the wearer.

Although several embodiments of the invention have been disclosed in the foregoing specification, it is understood by those skilled in the art that many modifications and other embodiments of the invention will come to mind to which the invention pertains, having the benefit of the teaching presented in the foregoing description and associated drawings. It is thus understood that the invention is not limited to the specific embodiments disclosed hereinabove, and that many modifications and other embodiments are intended to be included within the scope of the appended claims. Moreover, although specific terms are employed herein, as well as in the claims which follow, they are used only in a generic and descriptive sense, and not for the purposes of limiting the described invention, nor the claims which follow.

1. A wearable device for administering an electroretinography examination to a wearer of the device, the wearable device comprising:
a housing having a first side and a second side spaced apart relative to a transverse axis, the housing defining first and second compartments positioned along the transverse axis and having a ground electrode, each of the first and second compartments being configured for positioning over a respective eye of the wearer and comprising:
a stimulation light source,
a focal light source, wherein the focal light source is positioned at a location where the respective eye of the wearer is focused during administration of the electroretinography examination,
an active electrode that is configured to engage skin of the wearer, and
a reference electrode that is spaced from the active electrode and configured to engage skin of the wearer;
at least one processor communicatively coupled to the stimulation light source, the active electrode, and the reference electrode of each of the first and second compartments of the housing; and
a memory in communication with the processor, wherein the memory comprises instructions that, when executed by the at least one processor, cause the wearable device to:
cause the stimulation light source of the first compartment to flash; and
store a signal from the active electrode of the first compartment.

2. The wearable device of claim 1, wherein the memory comprises instructions that, when executed by the at least one processor, cause the at least one processor to detect at least one feature of the signal.

3. The wearable device of claim 2, wherein the memory comprises instructions that, when executed by the at least one processor, cause the at least one processor to determine a time delay between the flash of the stimulation light source and at least one feature of the signal.

4. The wearable device of claim 1, wherein the stimulation light sources of the first and second compartments are configured to uniformly illuminate an entire field of view of each eye of a wearer.

5. The wearable device of claim 1, wherein the stimulation light sources of the first and second compartments are configured to provide a dim flash having a single flash intensity.

6. The wearable device of claim 1, wherein the stimulation light sources of the first and second compartments are configured to provide a plurality of flashes of varying intensity.

7. The wearable device of claim 1, wherein the wearable device further comprises a head strap having a first end that is attached to the first side of the housing and a second end that is attached to the second side of the housing.

8. The wearable device of claim 1, wherein the housing comprises a flexible rim that is configured to conform to a face of the wearer.

9. The wearable device of claim 8, wherein the housing is configured to block out substantially all ambient light to eyes of the wearer.

10. The wearable device of claim 8, wherein the active electrodes and the reference electrodes of the first and second compartments and the ground electrode are embedded within the flexible rim.

11. The wearable device of claim 1, wherein the active electrode of the first compartment is positioned to engage skin of the wearer below the respective eye of the wearer.

12. The wearable device of claim 1, wherein the ground electrode is positioned to engage at least one of forehead skin or brow skin of the wearer.

13. The wearable device of claim 1, wherein the reference electrode of the first compartment is spaced further from a plane that is perpendicular to the transverse axis and bisects the housing between the first side and the second side than the active electrode of the first compartment.

14. The wearable device of claim 1, further comprising an output device, wherein the output device is a cable, a wireless transmitter, or an I/O port.

15. The wearable device of claim 1, wherein the housing defines a slot between the first and second compartments that is configured to conform to the shape of a nose of the wearer.

16. The wearable device of claim 1, wherein a spacing between the focal light sources of the first and second compartments is fixed.

17. The wearable device of claim 1, wherein a spacing between the focal light sources of the first and second compartments is selectively adjustable.

18. The wearable device of claim 1, wherein each of the first and second compartments comprises:
a peripheral interior wall that extends circumferentially around a respective eye of the wearer and projects away from the respective eye in a distal direction; and
a distal wall that extends between distal ends of the peripheral interior wall to enclose a space that is visible by the eye of the wearer,
wherein the stimulation light source and focal light source are secured to the distal wall.

19. A method of using the wearable device of claim 1, the method comprising:
positioning the wearable device over the eyes of a wearer;
executing instructions in the memory that cause the wearable device to perform a electroretinography test; and
receiving an output from the wearable device.

20. The method of claim 19, wherein executing instructions in the memory that cause the wearable device to perform an electroretinography test comprises simultaneously performing an electroretinography test on each eye of the wearer.

21. The method of claim 19, further comprising analyzing the output from the wearable device to determine whether the patient has diabetic retinopathy.

22. The method of claim 19, wherein the instructions are executed after an acclimation period of at least five minutes.

23. A wearable device for administering an electroretinography examination to a wearer of the device, the wearable device comprising:
a housing having a first side and a second side spaced apart relative to a transverse axis, the housing defining first and second compartments positioned along the transverse axis, each of the first and second compartments being configured for positioning over a respective eye of the wearer and comprising:
a stimulation light source,
a focal light source, wherein the focal light source is positioned at a location where the respective eye of the wearer is focused during administration of the electroretinography examination,
an active electrode that is configured to engage skin of the wearer, and
a reference electrode that is spaced from the active electrode and configured to engage skin of the wearer,
wherein the housing is configured to block substantially all ambient light from the eyes of the wearer.
